(19) United States
(12) Patent Application Publication
(10) Pub. No.: US 2024/0113675 A1
(43) Pub. Date: Apr. 4, 2024

(54) EARPHONE CONTROL METHOD, COMPUTER PROGRAM AND COMPUTER DEVICE

(71) Applicant: Olive Union, Inc., Seoul (KR)

(72) Inventors: Myunggeun Song, Seoul (KR); Taeho Son, Seoul (KR); Yoonsoo Kim, Seoul (KR); Donghyun Lee, Seoul (KR)

(21) Appl. No.: 18/303,664

(22) Filed: Apr. 20, 2023

(30) Foreign Application Priority Data
Oct. 4, 2022 (KR) 10 2022 0126244

(51) Int. Cl. H03G 3/32; H04R 1/10

(52) U.S. Cl. CPC H03G 3/32; H04R 1/1041

(57) ABSTRACT

Disclosed are an earphone control method, a computer program and a computer device. According to an embodiment, the earphone control method includes applying a user's minimum audible level information for each frequency band to an earphone, and controlling an output sound output by the earphone based on the applied minimum audible level information for each frequency band.

### CROSS-REFERENCE TO RELATED APPLICATIONS

A claim for priority under 35 U.S.C. § 119 is made to Korean Patent Application No. 10-2022-0126244 filed on Oct. 4, 2022, in the Korean Intellectual Property Office, the entire contents of which are hereby incorporated by reference.

### BACKGROUND

Embodiments of the inventive concept described herein relate to an earphone control method, a computer program, and a computer device.

With the recent leap development of biomedical engineering, patients who had not received much help from wearing a hearing aid in the past can select and wear an appropriate hearing aid, thereby achieving excellent hearing improvement.

The hearing aid is a state-of-the-art medical device that is always attached to a patient's body for use. The hearing aid needs to be continuously maintained according to changes in hearing and be managed through after-sales service for parts damaged by moisture and foreign substances in the ear. Therefore, the hearing aid is considered to be one of the most important technologies among biomedical engineering technologies.

In the past, the hearing aid takes the form of a trumpet-type sound collector, but currently the hearing aid usually takes the form of an electrical type that amplifies sound. The electrical hearing aid operates in such a way to receive sound waves through a microphone, convert the sound waves into electrical vibrations, amplify the electrical vibrations, and convert the electrical vibrations into sound waves back to enable the sound waves to be heard in ears. The prior art for the electrical hearing aid is described in Korean Patent Application Laid-Open No. 10-2013-0133790.

However, such a conventional electrical hearing aid has a limitation because of provision of merely a limited function.

Accordingly, there is a need to propose a device in which a function of a hearing aid and a function of an earphone are combined.

### SUMMARY

Embodiments provide a device in which a hearing aid function and an earphone function are combined.

Specifically, the embodiments propose an earphone, an earphone control platform, and a control method thereof for individually controlling an output volume of an environmental sound or a content sound for each frequency band by applying a user's minimum audible level information for each frequency band.

In this case, the embodiments propose an earphone, an earphone control platform, and a control method thereof for obtaining the user's minimum audible level information for each frequency band to be used for earphone control based on results of a hearing test through the earphone control platform.

In addition, the embodiments propose an earphone, an earphone control platform, and a control method for maximizing a user's convenience by initially applying the user's minimum audible level information for each frequency band to the earphone and then maintaining a state in which the minimum audible level information has been applied.

In addition, the embodiments propose an earphone, an earphone control platform, and a control method for controlling the application of the user's minimum audible level information for each frequency band to the earphone in real time according to a user input generated on the earphone control platform.

Further, embodiments of the inventive concept propose an earphone in which controlling of an output volume of an environmental sound for each frequency band individually, controlling of a degree of blocking of the output volume of the environmental sound for an entire frequency band on a step-wise basis, adjusting of the output volume of the environmental sound for the entire frequency band with respect to each of left and right units of the earphone, selectively removing of a feedback sound occurring in the process of outputting the output sound and controlling of the output volume of a content sound for each frequency band individually are supported on a single page, an earphone control platform, and a control method therefor.

In addition, the embodiments propose an earphone, an earphone control platform, and a control method thereof for measuring and reporting a noise level, a number of occurrences, and an occurrence time of the environmental sound in real time.

However, the technical problems to be solved by inventive concept are not limited to the above problems, and may be variously expanded without departing from the technical spirit and scope of inventive concept.

According to an exemplary embodiment, an earphone control method performed by a computer device including at least one processor includes applying a user's minimum audible level information for each frequency band to an earphone, and controlling an output sound output by the earphone based on the applied minimum audible level information for each frequency band.

The controlling of the output sound may include individually controlling an output volume of an environmental sound for each frequency band, the environmental sound being collected from an external environment of the earphone based on the applied minimum audible level information for each frequency band.

The controlling of the output sound may include individually controlling the output volume of the environmental sound for each frequency band based on the applied minimum audible level information for each frequency band while outputting a content sound related to content reproduced by a user terminal paired with the earphone, and outputting the environmental sound.

The controlling of the output sound may include controlling a degree of blocking of the output volume of the environmental sound for an entire frequency band on a step-wise basis based on a user input for controlling a degree of blocking, generated on an earphone control platform loaded into a user terminal paired with the earphone.

The controlling of the output sound may include adjusting the output volume of the environmental sound for an entire frequency band with respect to each of left and right units of the earphone based on a user input for performing an adjustment for each of the left and right units, the user input being generated on an earphone control platform loaded into a user terminal paired with the earphone.

The controlling of the output sound may include individually adjusting the output volume of the environmental sound for each frequency band further based on a user input for performing an adjustment for each frequency band, generated on an earphone control platform loaded into a user terminal paired with the earphone.

The applying of the minimum audible level information may include initially applying the user's minimum audible level information for each frequency band to the earphone once based on a user input for applying the minimum audible level information, generated on an earphone control platform and then maintaining a state in which the minimum audible level information has been applied.

The controlling of the output sound may include selectively removing a feedback sound occurring in outputting the output sound based on a user input for removing the feedback sound, generated on an earphone control platform loaded into a user terminal paired with the earphone.

The user's minimum audible level information may be obtained based on results of a user's hearing test for a plurality of frequency bands through an earphone control platform loaded into a user terminal paired with the earphone.

The controlling of the output sound may include individually controlling an output volume of a content sound related to content reproduced by a user terminal paired with the earphone based on the applied minimum audible level information for each frequency band.

The earphone control method may further include measuring a noise level, a number of occurrences, and an occurrence time of the output sound in real time, and reporting the noise level, the number of occurrences, and the occurrence time of the output sound measured in real time through an earphone control platform loaded into a user terminal paired with the earphone.

The measuring of the noise level, the number of occurrences, and the occurrence time of the output sound may include measuring in real time a noise level, a number of occurrences, and an occurrence time for each of the environmental sound collected from an external environment of the earphone and a content sound related to content reproduced by the user terminal paired with the earphone, and the reporting of the noise level, the number of occurrences, and the occurrence time of the output sound may include identifying and reporting the noise level, the number of occurrences, and the occurrence time for each of the environmental sound and the content sound.

The measuring of the noise level, the number of occurrences, and the occurrence time of the output sound may include providing an alarm to the user through the earphone control platform loaded into a user terminal paired with the earphone or an alarm sound output from the earphone when each of the noise level, the number of occurrences, and the occurrence time of the output sound measured in real time is equal to or greater than a corresponding preset threshold value.

According to an exemplary embodiment, there is provided a computer-readable recording medium recording a computer program causing a computer device to execute an earphone control method, wherein the earphone control method includes applying a user's minimum audible level information for each frequency band to an earphone, and controlling an output sound output by the earphone based on the applied minimum audible level information for each frequency band.

According to an exemplary embodiment, a computer device performing an earphone control method includes at least one processor configured to execute computer readable instructions, wherein the at least one processor may include an application unit that applies a user's minimum audible level information for each frequency band to an earphone, and a control unit that controls an output sound output by the earphone based on the applied minimum audible level information for each frequency band.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a diagram illustrating an example of a network environment according to an embodiment;

FIG. 2 is a block diagram illustrating an example of a computer device according to an embodiment;

FIG. 3 is a block diagram illustrating an example of components which may be included in a processor shown in FIG. 2;

FIG. 4 is a flowchart illustrating an earphone control method that the computer device shown in FIG. 2 is able to perform; and

FIGS. 5A to 5J are diagrams illustrating screens of a user terminal into which an earphone control platform is loaded, which is utilized when the earphone control method shown in FIG. 4 is performed.

### DETAILED DESCRIPTION

Hereinafter, embodiments of the inventive concept will be described in detail with reference to the exemplary drawings. However, it will be understood that the inventive concept is by no means restricted or limited in any manner by these embodiments. In addition, the same reference numeral shown in each drawing indicates the same component.

In addition, terminologies used in the present specification are used to properly express preferred embodiments of the inventive concept, and may be changed depending on the intention of viewers or operators, or customs in the field to which the inventive concept belongs. Accordingly, definitions of these terminologies should be made based on the content throughout this specification. For example, the singular expressions include plural expressions unless the context clearly dictates otherwise. Also, in this specification, the terms “comprises” and/or “comprising” are intended to specify the presence of stated features, integers, steps, operations, elements, parts or combinations thereof, but do not preclude the presence or addition of steps, operations, elements, parts, or combinations thereof.

Also, it should be understood that various embodiments of inventive concept are different from each other but are not necessarily mutually exclusive. For example, specific shapes, structures, and characteristics described herein may be implemented in other embodiments without departing from the spirit and scope of inventive concept in relation to one embodiment. In addition, it should be understood that the position, arrangement, or configuration of individual components may be changed in each embodiment without departing from the spirit and scope of inventive concept.

In the following embodiments, a device (hereinafter, the device is referred to as an “earphone”) in which a hearing aid function and an earphone function are combined, an earphone control platform used to control the device, and a control method thereof will be described.

The earphone may be controlled based on the earphone control platform to be described later, and an entity for controlling the earphone may be a server or an electronic device (user terminal) that performs the earphone control method.

The earphone control platform may be implemented in the form of a dedicated application installed in a user terminal paired with the earphone, and may operate as an interface for receiving a user input in the process of performing the earphone control method. The earphone control platform may operate on the user terminal under the control of a computer program installed and driven in at least one computer device constituting the user terminal.

The earphone control method may be performed by at least one computer device implementing a server or an electronic device (e.g., a user terminal) to be described later in response to a user input generated on the earphone control platform. That is, at least one computer device included in the server or electronic device, which will be described later, may constitute an earphone control system for performing the earphone control method. The computer device implementing the earphone control system may perform the earphone control method according to an embodiment under the control of the driven computer program. The above-described computer program may be stored in a computer-readable recording medium in combination with a computer device to allow to the computer device to execute an earphone control method. The computer program described herein may have the form of one independent program package, or the form of one independent program package may be pre-installed in a computer device and linked with an operating system or other program packages.

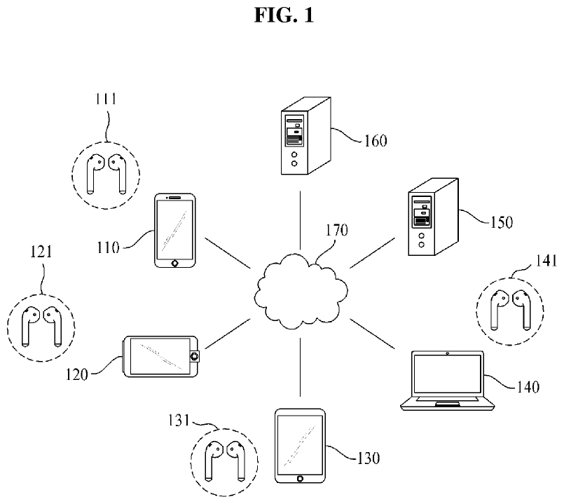

FIG. 1 is a diagram illustrating an example of a network environment according to an embodiment. The network environment of FIG. 1 shows an example including a plurality of electronic devices 110, 120, 130, and 140, a plurality of servers 150 and 160, and a network 170.

FIG. 1 is an example for describing the inventive concept, and the number of electronic devices or the number of servers is not limited as in FIG. 1. In addition, the network environment of FIG. 1 is only for describing one example of environments applicable to the present embodiments, and the environment applicable to the present embodiments is not limited to the network environment of FIG. 1.

The plurality of electronic devices 110, 120, 130, and 140 may be a fixed terminal implemented as a computer device or a mobile terminal. Examples of the plurality of electronic devices 110, 120, 130, and 140 include a smart phone, a mobile phone, a navigation device, a computer, a notebook computer, a digital broadcasting terminal, a personal digital assistant (PDA), a portable multimedia player (PMP), and a tablet PC. For example, although a smartphone is shown in FIG. 1 as an example of the electronic device 110, in embodiments, the electronic device 110 may refer to one of various physical computer devices capable of substantially communicating with other electronic devices 120, 130, and 140 and/or the server 150 or 160 through the network 170 using a wireless or wired communication method.

In particular, the electronic devices 110, 120, 130, and 140 are user terminals paired with earphones 111, 121, 131, and 141 through short-range wireless communication, respectively, and refer to devices in which an earphone control platform to be described later is driven.

Each of the earphones 111, 121, 131, and 141 may be controlled by an entity (e.g., the plurality of electronic devices 110, 120, 130, and 140 or the servers 150 and 160) performing an earphone control method based on the earphone control platform and may be a device having all of a hearing aid function (a function of outputting environmental sound collected from an external environment of a space where the earphone exists) and an earphone function (a function of outputting content sound related to content reproduced by the user terminal paired with the earphone). The hearing aid function and the earphone function may be operated individually, but are not limited thereto and may be operated simultaneously.

The communication method between the plurality of electronic devices 110, 120, 130, and 140 and the servers 150 and 160 is not limited, and may include not only a communication method using a communication network (e.g., a mobile communication network, a wired Internet, a wireless Internet, a broadcasting network) in which the network 170 may be included, but also short-range wireless communication between devices. For example, the network 170 may include one or more of networks, such as a personal area network (PAN), a local area network (LAN), a campus area network (CAN), a metropolitan area network (MAN), a wide area network (WAN), and a broadband network (BBN), the Internet, and the like. In addition, the network 170 may include any one or more of network topologies including a bus network, a star network, a ring network, a mesh network, a star-bus network, a tree or hierarchical network, and the like, but is not limited thereto.

Each of the servers 150 and 160 may communicate with the plurality of electronic devices 110, 120, 130, and 140 via the network 170 and may be implemented with a computer device or a plurality of computer devices that provides commands, codes, files, content, services, or the like. For example, the server 150 may be a system that controls and the earphones 111, 121, 131 and 141 through the earphone control platform installed in and driven on the plurality of electronic devices 110, 120, 130, and 140 connected through the network 170.

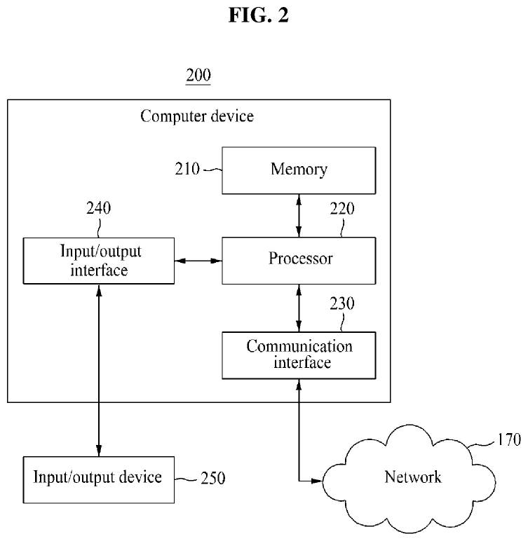

FIG. 2 is a block diagram illustrating an example of a computer device according to an embodiment. Each of the plurality of electronic devices 110, 120, 130, and 140 or each of the servers 150 and 160 which have been described above may be implemented by a computer device 200 illustrated in FIG. 2.

As shown in FIG. 2, the computer device 200 may include a memory 210, a processor 220, a communication interface 230, and an input/output interface 240. The memory 210 is a computer-readable recording medium and may include a random access memory (RAM), a read only memory (ROM), and a permanent mass storage device such as a disk drive. Here, a non-volatile mass storage device such as a ROM and a disk drive may be included in the computer device 200 as a separate permanent storage device that is distinct from the memory 210. Also, the memory 210 may store an operating system and at least one program code. These software components may be loaded into the memory 210 from a separate computer-readable recording medium distinct from the memory 210. The separate computer-readable recording medium may include a computer-readable recording medium such as a floppy drive, a disk, a tape, a DVD/CD-ROM drive, and a memory card. In another embodiment, the software components may be loaded into the memory 210 through a communication interface 230 rather than a computer-readable recording medium. For example, the software components may be loaded into the memory 210 of the computer device 200 based on a computer program installed by files received through the network 170.

The processor 220 may be configured to process instructions of a computer program by performing basic arithmetic, logic, and input/output operations. The instructions may be provided to the processor 220 by the memory 210 or the communication interface 230. For example, the processor 220 may be configured to execute a received instruction according to program codes stored in a recording device such as the memory 210.

The communication interface 230 may provide a function for allowing the computer device 200 to communicate with other devices (e.g., the storage devices described above) through the network 170. For example, a request, command, data, file, or the like generated by the processor 220 of the computer device 200 according to program codes stored in a recording device such as the memory 210 may be transmitted to other devices via the network 170. Conversely, a signal, command, data, file, or the like from another device may be received by the computer device 200 through the communication interface 230 of the computer device 200 via the network 170. A signal, command, data or the like received through the communication interface 230 may be transmitted to the processor 220 or the memory 210, and the file or the like may be stored in a storage medium (e.g., the persistent storage device described above) which may be further included in the computer device 200.

The input/output interface 240 may be means for interface with an input/output device 250. For example, the input device may include a device such as a microphone, keyboard, or mouse, and the output device may include a device such as a display or a speaker. As another example, the input/output interface 240 may be means for interface with a device in which an input function and an output function are integrated, such as a touch screen. The input/output device 250 may be configured as one device with the computer device 200.

Also, in other embodiments, the computer device 200 may include fewer or more components than those of FIG. 2. However, there is no need to clearly show most of the prior art components. For example, the computer device 200 may be implemented to include at least a part of the above-described input/output device 250 or may further include other components such as a transceiver and a database.

Hereinafter, specific embodiments of the earphone control method and system, and the earphone control platform will be described.

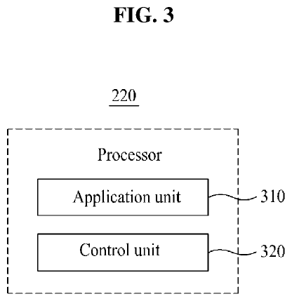

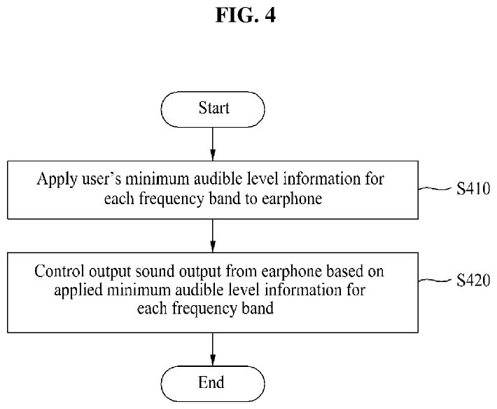

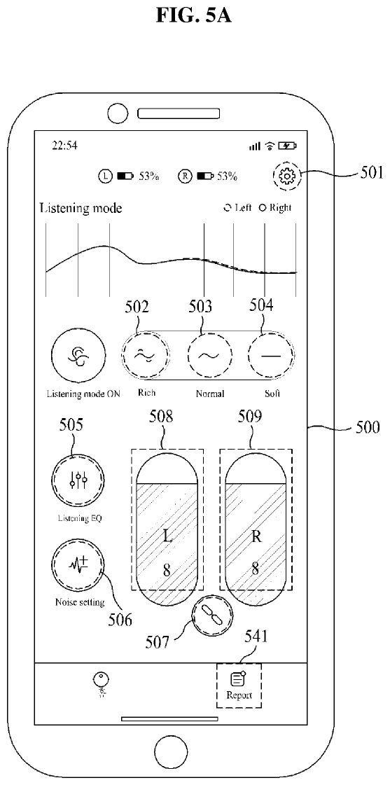

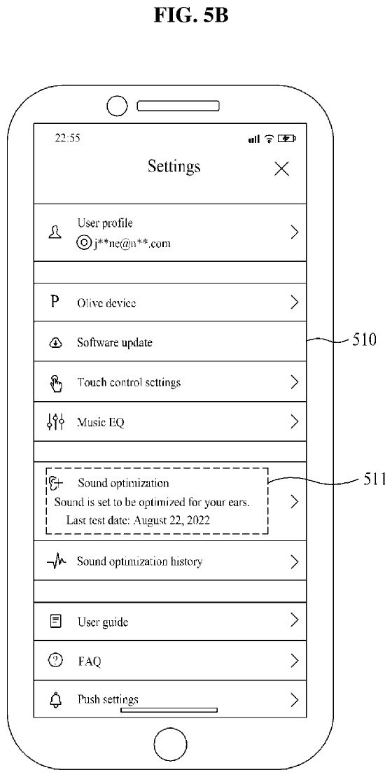

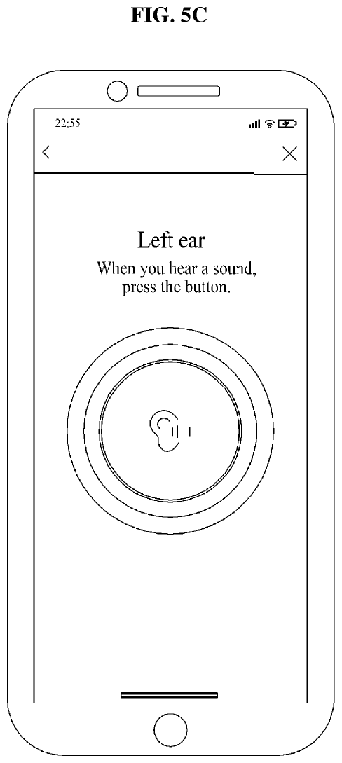

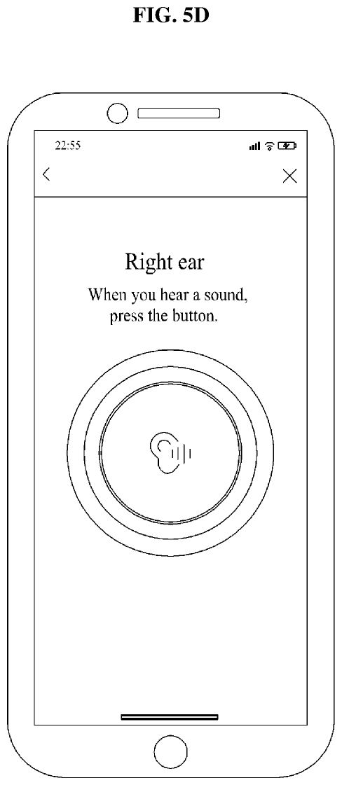

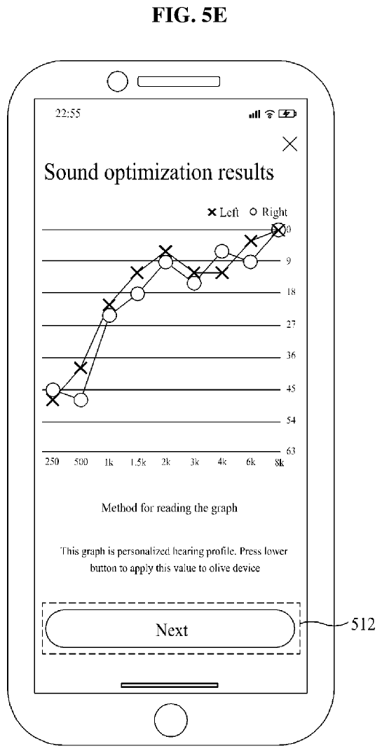

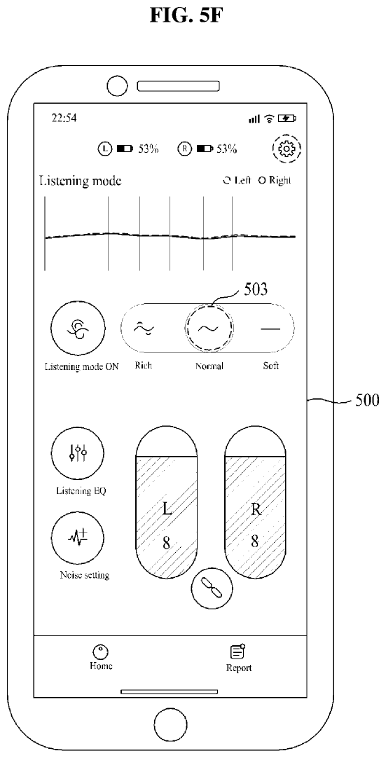

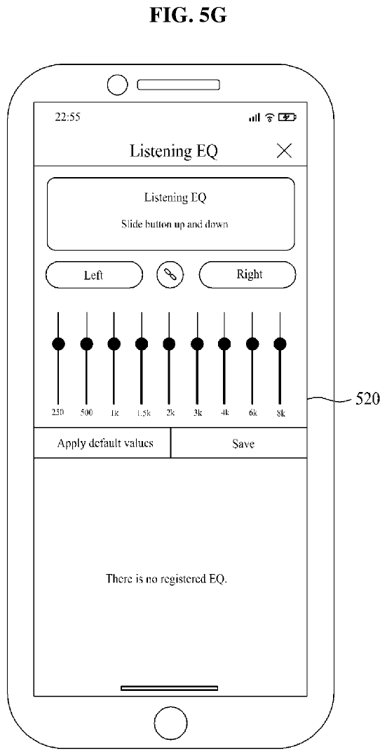

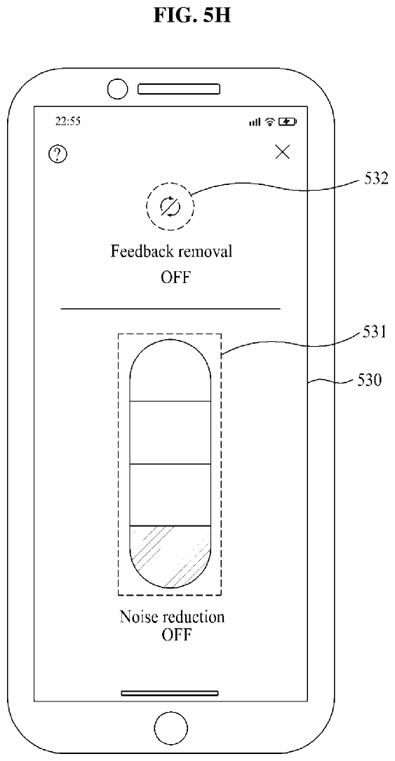

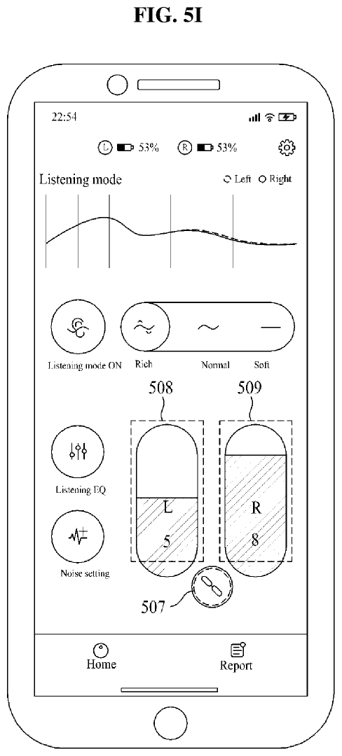

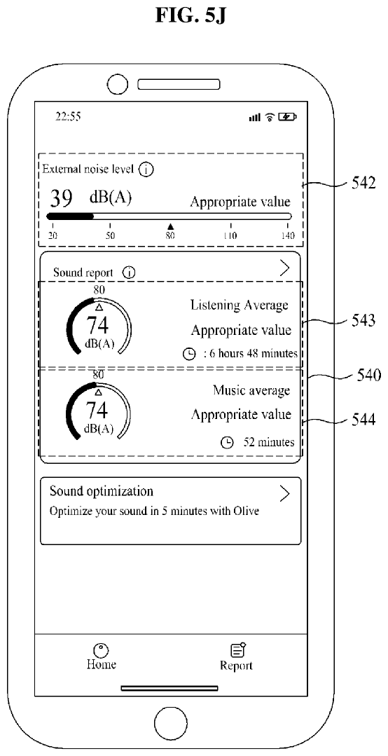

FIG. 3 is a block diagram illustrating an example of components which may be included in a processor shown in FIG. 2, FIG. 4 is a flowchart illustrating an earphone control method that the computer device shown in FIG. 2 is able to perform, and FIGS. 5A to 5J are diagrams illustrating screens of a user terminal into which an earphone control platform is loaded, which is utilized when the earphone control method shown in FIG. 4 is performed. Specifically, FIG. 5A is a view showing a main screen of an earphone control platform according to an embodiment, FIGS. 5B to 5E are diagrams for describing that a hearing test is performed through the earphone control platform according to an embodiment, FIG. 5F is a diagram for describing that an output volume for each frequency band of environmental sound is controlled based on a user's minimum audible level information for each frequency band through the earphone control platform according to the embodiment, FIG. 5G is a diagram for describing that an output volume for each frequency band of environmental sound is controlled based on a user input generated through the earphone control platform according to the embodiment, FIG. 5H is a diagram for describing that control of a degree of blocking of environmental sound and feedback removal are performed through the earphone control platform according to the embodiment, FIG. 5I is a diagram for describing that an output volume of an environmental sound is adjusted for each of left and right units of an earphone through the earphone control platform according to the embodiment, and FIG. 5J is a diagram for describing that information related to an environmental sound is reported through the earphone control platform according to the embodiment.

In embodiments of the inventive concept, the computer device 200 may perform the earphone control method to be described later in response to a user input generated through the earphone control platform, which is a dedicated application installed in the user terminal (e.g., the electronic device 110) to provide a service for controlling an earphone (e.g., the earphone 111 paired with the electronic device 110) to a user. To this end, the computer device 200 may be configured with an earphone control system, which is an entity that performs the earphone control method. For example, the earphone control system may be implemented in the form of a program that operates independently, or may be configured in the form of an in-app of a dedicated application to be able to operate on the dedicated application.

The processor 220 of the computer device 200 may be implemented as a component for performing the earphone control method according to FIG. 4. For example, the processor 220 may include an application unit 310 and a control unit 320 as show in in FIG. 3 to perform steps S410 to S420 shown in FIG. 4. Depending on embodiments, the components of the processor 220 may be selectively included in or excluded from the processor 220. Also, according to an embodiment, the components of the processor 220 may be separated from or merged into each other to express the functions of the processor 220.

The processor 220 and the components of the processor 220 may control the computer device 200 to perform steps S410 to S420 included in the earphone control method of FIG. 4. For example, the processor 220 and components of the processor 220 may be implemented to execute instructions according to the codes of an operating system included in the memory 210 and the codes of at least one program.

Here, the components of the processor 220 may be expressions of different functions performed by the processor 220 according to instructions provided by the program codes stored in the computer device 200. For example, the control unit 320 may be used as a functional representation of the processor 220 that controls the computer device 200 to control an output sound output to the user by the earphone.

The processor 220 may read a necessary instruction from the memory 210 in which instructions related to the control of the computer device 200 are loaded. In this case, the read instruction may include an instruction for allowing the processor 220 to execute steps S410 to S420 to be described later.

Steps S410 to S420 to be described later may be performed in an order different from that shown in FIG. 4, and some of steps S410 to S420 may be omitted or additional processes may be further included.

In step S410, the processor 220 (more specifically, the application unit 310 included in the processor 220) may apply the user's minimum audible level information for each frequency band to the earphone.

In this case, the user's minimum audible level information for each frequency band may be obtained based on the user's hearing test results for a plurality of frequency bands through the earphone control platform loaded into the user terminal paired with the earphone.

For example, referring to FIGS. 5A and 5B to 5E, when a user input is generated on a setting icon 501 of a main screen 500 of the earphone control platform, the processor 220 may load a setting screen 510 of the earphone control platform as shown in FIG. 5B. Subsequently, in response to a user input being generated on a sound optimization icon 511 of the setting screen 510 of the earphone control platform, the processor 220 may output a test sound while increasing or decreasing a volume for each frequency band with respect to each of the left and right units of the earphone and receive a user input indicating whether the user has heard the test sound through the earphone control platform as shown in FIGS. 5C and 5D to perform a hearing test for a user with respect to a plurality of frequency bands. The test sound may be output with the volume adjusted from 0 to 54 dB per frequency for 250 Hz, 500 Hz, 1 kHz, 1.5 kHz, 2 kHz, 3 kHz, 4 kHz, 6 kHz, and 8 kHz. The results obtained by performing the hearing test for the user may be provided to the user through the earphone control platform as shown in FIG. 5E. The user's minimum audible level information for each frequency band which is the provided results of the hearing test may be directly applied to the earphone in response to a user input for applying the minimum audible level information, generated on an application icon 512 as shown in FIG. 5E.

In this way, step S410 of applying the user's minimum audible level information for each frequency band to the earphone may mean that the user's minimum audible level information for each frequency band is initially applied to the earphone once based on the user input for applying the minimum audible level information, generated on the earphone control platform and a state in which the minimum audible level information has been applied is then maintained. To maintain the state in which the minimum audible level information has been applied may be continued as long as an individual control input of the output volume for each frequency band is not generated from the user in the earphone control platform.

In step S420, the processor 220 (more specifically, the control unit 320 included in the processor 220) may control an output sound output by the earphone based on the applied minimum audible level information for each frequency band through the step S410.

Here, the output sound may include an environmental sound collected from an external environment of the earphone and a content sound related to content reproduced by a user terminal paired with the earphone.

Specifically, the processor 220 may individually and in real time control the output volume of an environmental sound for each frequency band based on the applied minimum audible level information for each frequency band. For example, the processor 220 may individually and in real time control the output volume of the environmental sound for each frequency band in proportion to the user's minimum audible level information for each frequency band.

As another example, the processor 220 may control the output volume for each frequency band according to a preset listening mode under the condition that satisfies the minimum audible level information for each band in response to a user's selection input generated on any one of listening mode icons 502, 503 and 504 of the main screen 500 of the earphone control platform. As a more specific example, when the listening mode icon 502 corresponding to a “rich mode” in which the volume of a low-pitched frequency is increased compared to the volume of a high-pitched frequency is selected, the output volume of the environmental sound for each frequency band is controlled such that the volume of the low-pitched frequency is increased compared to the volume of the high-pitched frequency under the condition that satisfies the user's minimum audible level information for each frequency band. When the listening mode icon 503 corresponding to a “normal mode” in which the volume of the low-pitched frequency and the volume of the high-pitched frequency are uniform is selected, the output volume of the environmental sound for each frequency band is controlled such the volume of the low-pitched frequency and the volume of the high-pitched frequency are uniform under the condition that satisfies the user's minimum audible level information for each frequency band.

In this case, individual and real-time control of the output volume of the environmental sound for each frequency band based on the applied minimum audible level information for each frequency band may be performed regardless of whether the content sound is output. That is, the processor 220 may output content sound and, at the same time, individually and in real time control the output volume of an environmental sound for each frequency band based on the applied minimum audible level information for each frequency band. Accordingly, since the user can listen to the content sound and also the sound of the environmental sound whose output volume is controlled for each frequency band, the user may be freed from safety accidents caused by wearing earphones.

In addition, the processor 220 may control an output volume of the environmental sound for each frequency band further based on a user input for performing an adjustment for each frequency band, which is generated on the earphone control platform, without being limited or restricted to controlling the output volume of the environmental sound for each frequency band based on the applied user's minimum audible level information for each frequency band, and the user's frequency band adjustment generated by the earphone control platform.

For example, the processor 220 may load a listening EQ screen 520 as shown in FIG. 5G in response to a user input generated on the listening EQ (Equalizing) icon 505 of the main screen 500 of the earphone control platform, and receive the user's preferred volume for each of a plurality of frequency bands for each of the left and right units of the earphone to control the output volume of the environmental sound for each frequency band to be proportional to the received user input for performing an adjustment for each frequency band. In particular, the processor 220 may control the output volume of the environmental sound for each frequency band to be proportional to the user input for performing an adjustment for each frequency band under a condition that satisfies the user's minimum audible level information for each frequency band, thereby preventing hearing loss while satisfying the user's preferred volume for each frequency band.

Although the earphone control method in which the output volume of the environmental sound for each frequency band is individually controlled has been described above, the output volume of content sound for each frequency band may be individually controlled in the same way. For example, the processor 220 may individually control the output volume of the content sound for each frequency band based on the user's minimum audible level information for each frequency band.

In step S420, the processor 220 may not only control the output volume of the environmental sound for each frequency band, but also control a degree of blocking of the output volume of the environmental sound for the entire frequency band on a step-wise basis or selectively remove the feedback sound occurring in the process of outputting the environmental sound.

For example, the processor 220 may load a noise setting screen 530 as shown in FIG. 5H in response to a user input for making a selection, generated on a noise setting icon 506 of the main screen 500 of the earphone control platform and control a degree of blocking of the output volume of the environmental sound for the entire frequency band based on the user input for controlling a degree of blocking, generated on the loaded noise control icon 531 of the noise setting screen 530. The controlling of a degree of blocking of the output volume of the environmental sound for the entire frequency band may include steps for a first level (“noise reduction” OFF), a second level (“noise reduction” ON1), a third level (“noise reduction” ON2), a fourth level (“noise reduction” ON3).

As another example, the processor 220 may turn on or off a function for removing a feedback sound based on a user input for removing the feedback sound, generated on a feedback removal icon 532 of the noise setting screen 530 as shown in FIG. 5H. The processor 220 may not only remove the feedback of the environment sound, but also turn on or off the function of removing the feedback sound of content sound based on the user input for removing the feedback sound, generated on the feedback removal icon 532 when the content sound is being output.

In addition, in step S420, the processor 220 may not only control the output volume of the environmental sound for each frequency band, but also adjust the output volume of the environmental sound for the entire frequency band with respect to each left and right unit of the earphone. In addition, the output volume of the environmental sound for the entire frequency band may be collectively adjusted instead of being adjusted for each of the left and right units.

For example, the processor 220 may collectively adjust the output volume of the environmental sound for the entire frequency band at the same time with respect to the left and right units of the earphone when a user input for performing an adjustment is generated on either an earphone left unit icon 508 or an earphone right unit icon 509 while a batch control icon 507 of the main screen 500 of the earphone control platform is activated.

For another example, as shown in FIG. 5I, the processor 220 may adjust the output volume of the environmental sound for the entire frequency band with respect to an unit for which the input is generated when a user input for performing an adjustment is generated on either an earphone left unit icon 508 or an earphone right unit icon 509 while a batch control icon 507 of the main screen 500 of the earphone control platform is inactivated.

In addition, the processor 220 may measure a noise level, the number of occurrences, and an occurrence time of the output sound in real time and report the same to the user through the earphone control platform.

Specifically, the processor 220 may measure the noise level, the number of occurrences, and the occurrence time of the environmental sound and the content sound in real time, identify the noise level, the number of occurrences, and the occurrence time of each of the environmental sound and the content sound, and report the same to the user. For example, the processor 220 may load a report screen 540 as shown in FIG. 5J in response to a user input being generated on a report icon 541 on the main screen 500 of the earphone control platform and display and report, through the report screen 540, a real-time noise level 542 of the environmental sound, an occurrence time of the environmental sound, an average noise level 543 during the occurrence time and an occurrence time of the content sound, an average noise level 544 during the occurrence time to the user.

Here, the processor 220 may provide an alarm to the user through the earphone control platform or an alarm sound output from the earphone when each of the noise level, the number of occurrences, and the occurrence time of the output sound measured in real time is greater than or equal to a corresponding preset threshold value. Accordingly, the user may induce the user to protect their ears by selectively stopping the use of the earphone through the alarm.

As described above, controlling of the output volume of the environmental sound for each frequency band individually, controlling of a degree of blocking of the output volume of the environmental sound for the entire frequency band on a step-wise basis, adjusting of the output volume of the environmental sound for the entire frequency band with respect to each of left and right units of the earphone, selectively removing of a feedback sound occurring in the process of outputting the output sound and controlling of the output volume of a content sound for each frequency band individually may be performed in response to a user input generated on the main screen 500 of the earphone control platform. That is, the controlling of the output volume of the environmental sound for each frequency band individually, the controlling of a degree of blocking of the output volume of the environmental sound for the entire frequency band on a step-wise basis, the adjusting of the output volume of the environmental sound for the entire frequency band with respect to each of left and right units of the earphone, the selectively removing of a feedback sound occurring in the process of outputting the output sound and the controlling of the output volume of a content sound for each frequency band individually are supported on a single page of the earphone control platform, thereby maximizing the user's convenience in controlling the earphone.

The apparatus described herein may be implemented with hardware components and software components and/or a combination of the hardware components and the software components. For example, the apparatus and components described in the embodiments may be implemented using one or more general-purpose or special purpose computers, such as a processor, a controller and an arithmetic logic unit (ALU), a digital signal processor, a microcomputer, a field programmable array (FPGA), a programmable logic unit (PLU), a microprocessor or any other device capable of executing and responding to instructions. The processing device may run an operating system (OS) and one or more software applications that run on the OS. The processing device also may access, store, manipulate, process, and create data in response to execution of the software. For convenience of understanding, one processing device is described as being used, but those skilled in the art will appreciate that the processing device includes a plurality of processing elements and/or multiple types of processing elements. For example, the processing device may include multiple processors or a single processor and a single controller. In addition, different processing configurations are possible, such a parallel processors.

The software may include a computer program, a piece of code, an instruction, or some combination thereof, for independently or collectively instructing or configuring the processing device to operate as desired. Software and/or data may be embodied in any type of machine, component, physical equipment, computer storage medium or device that is capable of providing instructions or data to or being interpreted by the processing device. The software also may be distributed over network coupled computer systems so that the software is stored and executed in a distributed fashion. In particular, the software and data may be stored by one or more computer readable recording mediums.

The above-described methods may be embodied in the form of program instructions that can be executed by various computer means and recorded on a computer-readable medium. In this case, the medium may be to continuously store the program executable by the computer, or to temporarily store the program for execution or download. In addition, the medium may be various recording means or storage means in the form of a single or several hardware combined, and is not limited to a medium directly connected to any computer system, and may exist distributed on a network. Examples of the media include magnetic media such as hard disks, floppy disks and magnetic tape, optical media such as CD-ROMs, DVDs, and magneto-optical medium such as floptical disks, ROM, RAM, flash memory, and the like, which is specifically configured to store and execute program instructions. In addition, examples of other media include recording media or storage media managed by an app store that distributes applications, sites that supply or distribute various other software, and servers.

Although the embodiments have been described by the limited embodiments and the drawings as described above, various modifications and variations are possible to those skilled in the art from the above description. For example, the described techniques may be performed in a different order than the described method, and/or components of the described systems, structures, devices, circuits, etc. may be combined or combined in a different form than the described method, or other components, or even when replaced or substituted by equivalents, an appropriate result can be achieved.

Therefore, other implementations, other embodiments, and equivalents to the claims are within the scope of the following claims.

The embodiments may propose a device in which a hearing aid function and an earphone function are combined.

The embodiments may propose an earphone, an earphone control platform, and a control method thereof for individually controlling an output volume of an environmental sound or a content sound for each frequency band by applying a user's minimum audible level information for each frequency band.

In this case, the embodiments may propose an earphone, an earphone control platform, and a control method thereof for obtaining the user's minimum audible level information for each frequency band to be used for earphone control based on results of a hearing test through the earphone control platform.

In addition, the embodiments may propose an earphone, an earphone control platform, and a control method for maximizing a user's convenience by initially applying the user's minimum audible level information for each frequency band to the earphone and then maintaining a state in which the minimum audible level information has been applied.

In addition, the embodiments may propose an earphone, an earphone control platform, and a control method for controlling the application of the user's minimum audible level information for each frequency band to the earphone in real time according to a user input generated on the earphone control platform.

Further, embodiments of the inventive concept may propose an earphone in which controlling of an output volume of an environmental sound for each frequency band individually, controlling of a degree of blocking of the output volume of the environmental sound for an entire frequency band on a step-wise basis, adjusting of the output volume of the environmental sound for the entire frequency band with respect to each of left and right units of the earphone, selectively removing of a feedback sound occurring in the process of outputting the output sound and controlling of the output volume of a content sound for each frequency band individually are supported on a single page, an earphone control platform, and a control method therefor.

In addition, the embodiments may propose an earphone, an earphone control platform, and a control method thereof for measuring and reporting a noise level, a number of occurrences, and an occurrence time of the environmental sound in real time.

However, the effects of the inventive concept are not limited to the above effects, and may be variously expanded without departing from the spirit and scope of the inventive concept.

What is claimed is:

1. An earphone control method, the earphone control method being performed by a computer device including at least one processor, comprising:
applying a user's minimum audible level information for each frequency band to an earphone; and
controlling an output sound output by the earphone based on the applied minimum audible level information for each frequency band.

2. The earphone control method of claim 1, wherein the controlling of the output sound includes individually controlling an output volume of an environmental sound for each frequency band, the environmental sound being collected from an external environment of the earphone based on the applied minimum audible level information for each frequency band.

3. The earphone control method of claim 2, wherein the controlling of the output sound includes individually controlling the output volume of the environmental sound for each frequency band based on the applied minimum audible level information for each frequency band while outputting a content sound related to content reproduced by a user terminal paired with the earphone, and outputting the environmental sound.

4. The earphone control method of claim 2, wherein the controlling of the output sound includes controlling a degree of blocking of the output volume of the environmental sound for an entire frequency band on a step-wise basis based on a user input for controlling a degree of blocking, generated on an earphone control platform loaded into a user terminal paired with the earphone.

5. The earphone control method of claim 2, wherein the controlling of the output sound includes adjusting the output volume of the environmental sound for an entire frequency band with respect to each of left and right units of the earphone based on a user input for performing an adjustment for each of the left and right units, the user input being generated on an earphone control platform loaded into a user terminal paired with the earphone.

6. The earphone control method of claim 2, wherein the controlling of the output sound includes individually adjusting the output volume of the environmental sound for each frequency band further based on a user input for performing an adjustment for each frequency band, generated on an earphone control platform loaded into a user terminal paired with the earphone.

7. The earphone control method of claim 1, wherein the applying of the minimum audible level information includes initially applying the user's minimum audible level information for each frequency band to the earphone once based on a user input for applying the minimum audible level information, generated on an earphone control platform and then maintaining a state in which the minimum audible level information has been applied.

8. The earphone control method of claim 1, wherein the controlling of the output sound includes selectively removing a feedback sound occurring in outputting the output sound based on a user input for removing the feedback sound, generated on an earphone control platform loaded into a user terminal paired with the earphone.

9. The earphone control method of claim 1, wherein the user's minimum audible level information is obtained based on results of a user's hearing test for a plurality of frequency bands through an earphone control platform loaded into a user terminal paired with the earphone.

10. The earphone control method of claim 1, wherein the controlling of the output sound includes individually controlling an output volume of a content sound related to content reproduced by a user terminal paired with the earphone based on the applied minimum audible level information for each frequency band.

11. The earphone control method of claim 1, further comprising:
measuring a noise level, a number of occurrences, and an occurrence time of the output sound in real time; and
reporting the noise level, the number of occurrences, and the occurrence time of the output sound measured in real time through an earphone control platform loaded into a user terminal paired with the earphone.

12. The earphone control method of claim 11, wherein the measuring of the noise level, the number of occurrences, and the occurrence time of the output sound includes measuring in real time a noise level, a number of occurrences, and an occurrence time for each of the environmental sound collected from an external environment of the earphone and a content sound related to content reproduced by the user terminal paired with the earphone, and
wherein the reporting of the noise level, the number of occurrences, and the occurrence time of the output sound includes identifying and reporting the noise level, the number of occurrences, and the occurrence time for each of the environmental sound and the content sound.

13. The earphone control method of claim 11, wherein the measuring of the noise level, the number of occurrences, and the occurrence time of the output sound includes providing an alarm to the user through the earphone control platform loaded into a user terminal paired with the earphone or an alarm sound output from the earphone when each of the noise level, the number of occurrences, and the occurrence time of the output sound measured in real time is equal to or greater than a corresponding preset threshold value.

14. A computer-readable recording medium recording a computer program causing a computer device to execute an earphone control method,
wherein the earphone control method includes:
applying a user's minimum audible level information for each frequency band to an earphone; and
controlling an output sound output by the earphone based on the applied minimum audible level information for each frequency band.

15. A computer device performing an earphone control method comprising:
at least one processor configured to execute computer readable instructions,
wherein the at least one processor includes:
an application unit configured to apply a user's minimum audible level information for each frequency band to an earphone; and
a control unit configured to control an output sound output by the earphone based on the applied minimum audible level information for each frequency band.
